SEQUENCE LISTING (34 sequences)

SEQ ID NO: 1 - DNA, 20 bp, synthetic construct
  source 1..20: mol_type = other DNA
gttctgggattccgaggttg

SEQ ID NO: 2 - DNA, 21 bp, synthetic construct
  source 1..21: mol_type = other DNA
acttcgtcgcagctcatctga

SEQ ID NO: 3 - DNA, 23 bp, synthetic construct
  source 1..23: mol_type = other DNA
cggacttgtaacgaatccaaact

SEQ ID NO: 4 - DNA, 21 bp, synthetic construct
  source 1..21: mol_type = other DNA
ggagcatttatgtcccggaaa

SEQ ID NO: 5 - DNA, 23 bp, synthetic construct
  source 1..23: mol_type = other DNA
acagcgacctggtctactgaatc

SEQ ID NO: 6 - DNA, 20 bp, synthetic construct
  source 1..20: mol_type = other DNA
ggcttgaatcgtgtgttcca

SEQ ID NO: 7 - DNA, 23 bp, synthetic construct
  source 1..23: mol_type = other DNA
gctgccaataggtcaatatggaa

SEQ ID NO: 8 - DNA, 20 bp, synthetic construct
  source 1..20: mol_type = other DNA
tttcgctggttgctctttca

SEQ ID NO: 9 - DNA, 20 bp, synthetic construct
  source 1..20: mol_type = other DNA
aacagcgccagaaaaaaagg

SEQ ID NO: 10 - DNA, 26 bp, synthetic construct
  source 1..26: mol_type = other DNA
ggaaaggtagaaggtgaaccataaaa

SEQ ID NO: 11 - DNA, 24 bp, synthetic construct
  source 1..24: mol_type = other DNA
gagatggaaagattggcaacagcg

SEQ ID NO: 12 - DNA, 24 bp, synthetic construct
  source 1..24: mol_type = other DNA
agtgatgttaggctcagtggggac

SEQ ID NO: 13 - DNA, 25 bp, synthetic construct
  source 1..25: mol_type = other DNA
aattctcctttgtcaattccggccc

SEQ ID NO: 14 - DNA, 24 bp, synthetic construct
  source 1..24: mol_type = other DNA
ccagtactttatcgggtcccactg

SEQ ID NO: 15 - DNA, 19 bp, synthetic construct
  source 1..19: mol_type = other DNA
cagggcggcggtcgagctc

SEQ ID NO: 16 - DNA, 29 bp, synthetic construct
  source 1..29: mol_type = other DNA
ctttcgtgccgtagaagtctgtcattcca

SEQ ID NO: 17 - DNA, 16 bp, synthetic construct
  source 1..16: mol_type = other DNA
ccgctaacaggtcact

SEQ ID NO: 18 - DNA, 17 bp, synthetic construct
  source 1..17: mol_type = other DNA
catacgattcaagccac

SEQ ID NO: 19 - DNA, 15 bp, synthetic construct
  source 1..15: mol_type = other DNA
ccaaagaggaaaatc

SEQ ID NO: 20 - DNA, 78 bp, synthetic construct
  source 1..78: mol_type = other DNA
cggacttgtaacgaatccaaacttctttcgtgccgtagaagtctgtcattccagttttttccgggacataaatgctcc

SEQ ID NO: 21 - DNA, 78 bp, synthetic construct
  source 1..78: mol_type = other DNA
ggagcatttatgtcccggaaaaaactggaatgacagacttctacggcacgaaagaagtttggattcgttacaagtccg

SEQ ID NO: 22 - DNA, 61 bp, synthetic construct
  source 1..61: mol_type = other DNA
acagcgacctggtctactgaatcgccgctaacaggtcactatggaacacacgattcaagcc

SEQ ID NO: 23 - DNA, 61 bp, synthetic construct
  source 1..61: mol_type = other DNA
ggcttgaatcgtgtgttccatagtgacctgttagcggcgattcagtagaccaggtcgctgt

SEQ ID NO: 24 - DNA, 62 bp, synthetic construct
  source 1..62: mol_type = other DNA
gctgccaataggtcaatatggaacatacgattcaagccacggtgaaagagcaaccagcgaaa

SEQ ID NO: 25 - DNA, 62 bp, synthetic construct
  source 1..62: mol_type = other DNA
tttcgctggttgctctttcaccgtggcttgaatcgtatgttccatattgacctattggcagc

SEQ ID NO: 26 - DNA, 66 bp, synthetic construct
  source 1..66: mol_type = other DNA
aacagcgccagaaaaaaaggaccccaaagaggaaaatcaattttatggttcaccttctacctttcc

SEQ ID NO: 27 - DNA, 66 bp, synthetic construct
  source 1..66: mol_type = other DNA
ggaaaggtagaaggtgaaccataaaattgattttcctctttggggtcctttttttctggcgctgtt

SEQ ID NO: 28 - DNA, 72 bp, synthetic construct
  source 1..72: mol_type = other DNA
ttgttgttctgggattccgaggttgccaggcggcggtcgagctctctcagatgagctgcgacgaagtgatac

SEQ ID NO: 29 - DNA, 90 bp, synthetic construct
  source 1..90: mol_type = other DNA
gagaacggacttgtaacgaatccaaacttctttcgtgccgtagaagtctgtcattccagttttttccgggacataaatgctccaaagcag

SEQ ID NO: 30 - DNA, 69 bp, synthetic construct
  source 1..69: mol_type = other DNA
caaatacagcgacctggtctactgaatcgccgctaacaggtcactatggaacacacgattcaagccatg

SEQ ID NO: 31 - DNA, 72 bp, synthetic construct
  source 1..72: mol_type = other DNA
gaagcgctgccaataggtcaatatggaacatacgattcaagccacggtgaaagagcaaccagcgaaaacaga

SEQ ID NO: 32 - DNA, 80 bp, synthetic construct
  source 1..80: mol_type = other DNA
caatacaacagcgccagaaaaaaaggaccccaaagaggaaaatcaattttatggttcaccttctacctttccttacagag

SEQ ID NO: 33 - DNA, 62 bp, synthetic construct
  source 1..62: mol_type = other DNA
catgggagatggaaagattggcaacagcgggtaagtccccactgagcctaacatcactatta

SEQ ID NO: 34 - DNA, 65 bp, synthetic construct
  source 1..65: mol_type = other DNA
ggtaaaattctcctttgtcaattccggcccgggcggcagtgggacccgataaagtactggctgaa

(19) United States
(12) Patent Application Publication
(10) Pub. No.: US 2025/0376737 A1
(43) Pub. Date: Dec. 11, 2025

(54) QUANTITATIVE PCR ASSAYS FOR DETECTION OF PORCINE VIRAL RNAS AFTER XENOTRANSPLANT

(71) Applicant: University of Maryland, Baltimore, Baltimore, MD (US)

(72) Inventors: Alip Ghosh, Baltimore, MD (US); Kapil Saharia, Chevy Chase, MD (US); Ki Seok Lee, Timonium, MD (US); Shyamasundaran Kottilil, Timonium, MD (US)

(21) Appl. No.: 19/186,559

(22) Filed: Apr. 22, 2025

Related U.S. Application Data
(60) Provisional application No. 63/637,170, filed on Apr. 22, 2024.

(51) Int. Cl. C12Q 1/70

(52) U.S. Cl. CPC C12Q 1/701

(57) ABSTRACT

The present invention provides methods and kits for detecting and quantifying the presence of porcine virus PERV-A, PERV-B, PCMV, PCV3, PLHV-1, PLHV-2, and PLHV-3 nucleic acids in a sample from a xenotransplant subject that has received cells, tissues or an organ from a pig.

### CROSS-REFERENCE TO RELATED APPLICATIONS

This application claims the benefit of U.S. Application No. 63/637,170, filed Apr. 22, 2024, the contents of which are incorporated by reference in their entirety.

### INCORPORATION-BY-REFERENCE OF MATERIAL SUBMITTED ELECTRONICALLY

Incorporated by reference in its entirety herein is a computer-readable sequence listing and identified as follows: One 31,768 Byte XML file named “Sequence_listing.xml,” created on Apr. 17, 2025.

### FIELD OF THE INVENTION

The present invention generally relates to the fields of molecular biology, virology, medicine and transplantation. In particular, the field of the invention relates to xenotransplantation.

### BACKGROUND

The concept of xenotransplant became popular with increasing demand and shortage of the supply of human organs for transplant recipients. Advances in genetic engineering improved the survival of cardiac xenografts in the genetically engineered pig (GEP)-to-nonhuman primate (NHP) transplantation (Mohiuddin et al., Am J Transplant, (2012), 12:763-71; Mohiuddin et al., J Thorac Cardiovasc Surg, (2014), 148:1106-13: discussion 13-4). Success in GEP-to-NHP xenotransplant made it possible to take a leap to test GEP-to-human cardiac xenotransplant in terminally ill recipients for the first time in 2022 and a second time in 2023 at the University of Maryland, Baltimore, USA (Griffith et al., N Engl J Med, (2022), 387:35-44). In both cases hyperacute rejection did not occur and the recipients survived several weeks post cardiac xenotransplant (Griffith et al., N Engl J Med, (2022), 387:35-44). These success stories laid the possibility of future clinical trials to use porcine organs/cells for human recipients.

Although the potential benefits are considerable, a major concern in the use of porcine xenotransplant is potential infection of the recipients with infectious porcine viruses and the possible subsequent transmission to their close relatives, health care providers closely working with the recipients and eventually into the general human population (Kimsa et al., Viruses, (2014), 6:2062-83). Since cross-species infection by retroviruses can have a long latency and lead to disease years after infection, all these at-risk individuals may require long-term surveillance for the presence of porcine viral RNA in the blood (Gessain et al., Virology, (2013), 435:187-99).

Accordingly, there is a need for a highly sensitive and robust assay and kits for detecting and quantifying porcine viruses in subjects following xenotransplantation.

### SUMMARY OF THE INVENTION

It is to be understood that both the foregoing general description of the embodiments and the following detailed description are exemplary, and thus do not restrict the scope of the embodiments.

The present invention provides methods and kits for surveillance of porcine viral pathogens in human xenotransplant recipients. The present invention provides a robust and highly sensitive testing procedure to detect and quantify PERV-A, PERV-B, PCMV, PCV3, PLHV-1, PLHV-2, and PLHV-3, which are all porcine viruses, for example, in human plasma and peripheral blood mononuclear cell samples.

In one aspect, the invention provides a method of detecting and quantifying the presence of porcine viruses in a sample from a xenotransplant subject, comprising i) providing a sample from the xenotransplant subject, wherein the subject has received cells, tissues or an organ from a pig; ii) detecting the presence or absence of porcine virus nucleic acid in the sample that is amplified using a polymerase chain reaction (PCR) assay; and iii) quantifying porcine virus nucleic acid detected in the sample, wherein the porcine virus comprises one or more of PERV-A, PERV-B, PCMV, PCV3, PLHV-1, PLHV-2, and PLHV-3.

In another aspect, the invention provides a kit for detecting and quantifying the presence of porcine viruses in a sample from a xenotransplant subject, wherein the subject has received cells, tissues or an organ from a pig, comprising: i) one or more sets of oligonucleotide primers comprising a forward and reverse primer for amplifying porcine virus nucleic acid in a sample using a PCR assay; ii) one or more control templates comprising the porcine virus nucleic acid; iii) optionally one or more probes and/or dyes for detecting and quantifying the amplified porcine virus nucleic acid; and iv) optionally one or more reagents for performing PCR, wherein the porcine virus comprises one or more of PERV-A, PERV-B, PCMV, PCV3, PLHV-1, PLHV-2, and PLHV-3.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The skilled artisan will understand that the drawings, described below, are for illustration purposes only. The drawings are not intended to limit the scope of the present teachings in any way.

FIG. 1. Gel-electrophoresis picture showing bands of amplified products in semi-quantitative RT-PCR to determine the specificity and cross-reactivity of the primer sets. The 1st lane of the gel contains DNA marker (M) with known band sizes. Lane 2-8 represents amplified PCR products from respective positive controls indicating specificity of the primers. Lane 9-15 represents analysis of cross-reactivity, where templet in the PCR-mix included all positive controls and human gDNA except the positive control of the corresponding primer sets used in the mix as described by the table below.

FIG. 2. Linearity ranges of the real-time qPCR assays for porcine viruses (A) PERV-A, (B) PERV-B, (C) PCMV, (D) PCV3, (E) PLHV-1, (F) PLHV-2 and (G) PLHV-3. Mean values of the observed copy numbers, determined by standard curves, were plotted against the expected (known) copy numbers and Pearson's correlation were determined. Mean and standard deviations of 9 independent observations were plotted for each data point in each analysis. r=correlation coefficient.

FIG. 3. Sequence alignments for PCMV targeted primer and probe sequences in MAS viewer from blast analysis. Each row represents publicly available sequences with accession numbers (first column) and name of the organisms (last column). The sequences corresponding to the primers and the probe were marked by black frames. The sequence mismatches were marked in red.

FIG. 4. Sequence alignments for PCV3 targeted primer and probe sequences in MAS viewer from blast analysis. Each row represents publicly available sequences with accession numbers (first column) and name of the organisms (last column). The sequences corresponding to the primers and the probe were marked by black frames. The sequence mismatches were marked in red.

FIG. 5. Sequence alignments for PLHV-1 targeted primer and probe sequences in MAS viewer from blast analysis. Each row represents publicly available sequences with accession numbers (first column) and name of the organisms (last column). The sequences corresponding to the primers and the probe were marked by black frames. The sequence mismatches were marked in red.

FIG. 6. Sequence alignments for PLHV-2 targeted primer and probe sequences in MAS viewer from blast analysis. Each row represents publicly available sequences with accession numbers (first column) and name of the organisms (last column). The sequences corresponding to the primers and the probe were marked by black frames. The sequence mismatches were marked in red.

FIG. 7. Sequence alignments for PL HV-3 targeted primer and probe sequences in MAS viewer from blast analysis. Each row represents publicly available sequences with accession numbers (first column) and name of the organisms (last column). The sequences corresponding to the primers and the probe were marked by black frames. The sequence mismatches were marked in red.

FIG. 8. Sequence alignments for PERV-A targeted primer sequences in MAS viewer from blast analysis. Each row represents publicly available sequences with accession numbers (first column) and name of the organisms (last column). The sequences corresponding to the primers and the probe were marked by black frames. The sequence mismatches were marked in red.

FIG. 9. Sequence alignments for PERV-B targeted primer sequences in MAS viewer from blast analysis. Each row represents publicly available sequences with accession numbers (first column) and name of the organisms (last column). The sequences corresponding to the primers and the probe were marked by black frames. The sequence mismatches were marked in red.

### DETAILED DESCRIPTION

This disclosure provides a robust and highly sensitive real-time qPCR assay and kits for the detection and quantitation of porcine cytomegalovirus (PCMV), porcine circovirus (PCV3), porcine lymphotropic herpesviruses (PLHV-1, PLHV-2, and PLHV-3) and porcine endogenous retroviruses (PERV-A and PERV-B) RNA in human plasma and PBMC samples using developed positive controls. The applicability of the assays was evaluated in the clinical samples of two cardiac xenotransplant recipients.

As described herein, the lower limits of detection (LLoD) in human PBMCs were 1.25 copies/ng total RNA for PERV-A, PERV-B and PLHV-2, and 0.5 copies/ng total RNA for PCMV, PCV3, PLHV-1 and PLHV-3. The LLOD in human plasma specimens were 2 copies/μl of plasma for PERV-A, PERV-B and PLHV-2 and 0.8 copies/μl of plasma for PCMV, PCV3, PLHV-1 and PLHV-3. All the assays have high reproducibility in the linear range of 10¹-10⁸ copies. The assays show high specificity for respective target viral RNAs. The copy numbers of all these viruses have been quantified in one donor pig, and two porcine cardiac xenotransplant recipients. PERV-A and PERV-B RNAs were detected in low quantity from various tissues of the donor pig, while RNA quantity of all the other porcine viruses were below LLOD. The assay revealed no transmission of any of the porcine viruses to the recipients as copy numbers of all the viral RNAs were bellow LLOD in both plasma and PBMCs specimens collected one week after and one month after the xenotransplants. Thus, the real-time qPCR assay described herein is robust and highly specific to detect and quantify porcine PERV-A, PERV-B, PCMV, PCV3, PLHV-1, PLHV-2, and PLHV-3 viral RNAs in plasma and PB M C specimens. These assays can be used reliably for surveillance of possible transmission of porcine viruses to human in GEP-to-human xenotransplant.

Reference will now be made in detail to embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

One skilled in the art may refer to general reference texts for detailed descriptions of known techniques discussed herein or equivalent techniques. These texts include Current Protocols in Molecular Biology (A Usubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341).

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means at most plus or minus 10% of the numerical value of the number with which it is being used.

In one embodiment, the invention provides a method of detecting and quantifying the presence of porcine viruses in a sample from a xenotransplant subject, comprising,
i) providing a sample from the xenotransplant subject, wherein the subject has received cells, tissues or an organ from a pig;
ii) detecting the presence or absence of porcine virus nucleic acid in the sample that is amplified using a polymerase chain reaction (PCR) assay; and
iii) quantifying porcine virus nucleic acid detected in the sample,
wherein the porcine virus comprises one or more of PERV-A, PERV-B, PCMV, PCV3, PLHV-1, PLHV-2, and PLHV-3.

In some embodiments, the cells, tissue or organ from the pig have been genetically modified. The genetic modification is not necessarily limiting. Unmodified wild-type non-human animal tissues can be rejected by recipients, such as humans, by the immune system. Rejection is believed to be caused at least in part by antibodies binding to the tissues and cell-mediated immunity leading to graft loss. For example, pig grafts can be rejected by cellular mechanisms mediated by adaptive immune cells. In some embodiments, the cells, tissue or organ have been genetically modified to attenuate rejection by the subject. The method of any of claims 1-29, wherein the subject has received a genetically modified pig heart. See, e.g., WO 2016/094679.

The method comprises assaying for the presence of absence of nucleic acid from any of PERV-A, PERV-B, PCMV, PCV3, PLHV-1, PLHV-2, or PLHV-3. In some embodiments the presence of absence of nucleic acid from PERV-A is assayed. In some embodiments the presence of absence of nucleic acid from PERV-B is assayed. In some embodiments the presence of absence of nucleic acid from PCMV is assayed. In some embodiments the presence of absence of nucleic acid from PCV3 is assayed. In some embodiments nucleic acid from PLHV-1 is assayed. In some embodiments the presence of absence of nucleic acid from PLHV-2 is assayed. In some embodiments the presence of absence of nucleic acid from PERV-A is assayed. In some embodiments the presence of absence of nucleic acid from PLHV-3 is assayed. In some embodiments the presence of absence of nucleic acid from at least two porcine viruses is assayed. In some embodiments the presence of absence of nucleic acid from at least three porcine viruses is assayed. In some embodiments the presence of absence of nucleic acid from at least four porcine viruses is assayed. In some embodiments the presence of absence of nucleic acid from at least five porcine viruses is assayed. In some embodiments the presence of absence of nucleic acid from at least six porcine viruses is assayed. In some embodiments the presence of absence of nucleic acid from at least seven porcine viruses is assayed. In some embodiments the presence of absence of nucleic acid from PERV-A, PERV-B, PCMV, PCV3, PLHV-1, PLHV-2, and PLHV-3 is assayed.

As used herein, the term “subject” is not limiting and is used interchangeably with patient. In some embodiments, the term subject refers to animals, such as mammals and the like. For example, mammals contemplated include humans, primates, dogs, sheep, cattle, goats, horses and the like. In some embodiments, the subject is a human.

The sample from the subject to be assayed is not limiting. In some embodiments, the sample is blood, plasma or isolated cells. The sample can be processed in various ways prior to its being assayed for the presence of absence of porcine nucleic acid. For example, the sample can be isolated, fractionated, purified, etc., in order to isolate or purify cells, nucleic acids, etc., from the sample for further processing and PCR. In some embodiments, the sample is selected from the group consisting of blood, plasma, and peripheral blood mononuclear cells (PBMCs).

In some embodiments, the nucleic acid is RNA. In some embodiments, the nucleic acid is DNA. The polymerase chain reaction assay is not particularly limiting. In some embodiments, in the case where the starting nucleic acids in the sample are RNA, the RNA is converted to cDNA using an enzyme, such as reverse transcriptase, followed by amplification cycles of the cDNA using a suitable polymerase.

The number of cycles of PCR or the parameters are not necessarily limiting. In some embodiments, no more than 36 cycles of PCR are performed.

In some embodiments, the nucleic acid is detected and quantified using dyes and/or nucleic acid probes. In some embodiments, the nucleic acid is detected and quantified using a dye, such as a fluorescent dye, that binds to the porcine virus nucleic acid, such as SY BR Green dye (ThermoFisher Scientific). Reagents suitable for carrying out PCR amplification include buffer, dNTPs, thermostable hot-start DNA polymerase, for example. In some embodiments, the nucleic acid is detected and quantified using a labeled nucleic acid probe that binds to the nucleic acid. In some embodiments the probe is labeled with a fluorescent dye. See, e.g., TaqM an probes (ThermoFisher Scientific).

In some embodiments, copy number of the porcine virus nucleic acid in the sample can be quantified. In some embodiments, the copy number is achieved by using a standard curve generated using amplified control nucleic acid, and then comparing the quantified amplified nucleic acid from the sample to the standard curve and determining the copy number. In some embodiments, the amplified control nucleic acid is generated by polymerase chain reaction with a template comprising nucleic acid of a known quantity. In some embodiments, the template is a plasmid template comprising porcine virus nucleic acid of a known quantity, e.g. in effect the same nucleic acid fragment that would be amplified using the oligonucleotide primers to amplify the porcine nucleic acid in the sample from the subject.

In some embodiments, the amplified PERV-A and PERV-B nucleic acid is quantified using a fluorescent dye such as a SY BR Green dye that binds to the nucleic acid. In some embodiments, the amplified PCMV, PCV3, PLHV-1, PLHV-2, and PLHV-3 nucleic acid is quantified using a nucleic acid probe labeled with a fluorescent dye, such as a TaqM an probe.

In some embodiments, at a detection rate of 100%, the lower limit of detection of the copy number of PERV-A, PERV-B and PLHV-2 is about 25 copies/μL determined using positive controls. In some embodiments, at a detection rate of 100%, the lower limit of detection of the copy number of PERV-A, PERV-B and PLHV-2 is about 50 copies/μL. In some embodiments, at a detection rate of 100%, the lower limit of detection of the copy number of PERV-A, PERV-B and PLHV-2 is about 75 copies/μL. In some embodiments, at a detection rate of 100%, the lower limit of detection of the copy number of PERV-A, PERV-B and PLHV-2 is about 100 copies/μL.

In some embodiments, at a detection rate of 100%, the lower limit of detection of the copy number of PCMV, PCV3, PLHV-1 and PLHV-3 is about 10 copies/μL. In some embodiments, at a detection rate of 100%, the lower limit of detection of the copy number of PCMV, PCV3, PLHV-1 and PLHV-3 is about 25 copies/μL. In some embodiments, at a detection rate of 100%, the lower limit of detection of the copy number of PCMV, PCV3, PLHV-1 and PLHV-3 is about 50 copies/μL. In some embodiments, at a detection rate of 100%, the lower limit of detection of the copy number of PCMV, PCV3, PLHV-1 and PLHV-3 is about 100 copies/μL.

In some embodiments, the PCMV nucleic acid is amplified using a forward oligonucleotide primer comprising SEQ ID NO:1 or a portion thereof and a reverse oligonucleotide primer comprising SEQ ID NO:2 or a portion thereof.

In some embodiments, the PCV nucleic acid is amplified using a forward oligonucleotide primer comprising SEQ ID NO:3 or a portion thereof and a reverse oligonucleotide primer comprising SEQ ID NO:4 or a portion thereof.

In some embodiments, the PLHV-1 nucleic acid is amplified using a forward oligonucleotide primer comprising SEQ ID NO:5 or a portion thereof and a reverse oligonucleotide primer comprising SEQ ID NO:6 or a portion thereof.

In some embodiments, the PLHV-2 nucleic acid is amplified using a forward oligonucleotide primer comprising SEQ ID NO:7 or a portion thereof and a reverse oligonucleotide primer comprising SEQ ID NO:8 or a portion thereof.

In some embodiments, the PLHV-3 nucleic acid is amplified using a forward oligonucleotide primer comprising SEQ ID NO:9 or a portion thereof and a reverse oligonucleotide primer comprising SEQ ID NO:10 or a portion thereof.

In some embodiments, the PERV-A nucleic acid is amplified using a forward oligonucleotide primer comprising SEQ ID NO:11 or a portion thereof and a reverse oligonucleotide primer comprising SEQ ID NO:12 or a portion thereof.

In some embodiments, the PERV-B nucleic acid is amplified using a forward oligonucleotide primer comprising SEQ ID NO:13 or a portion thereof and a reverse oligonucleotide primer comprising SEQ ID NO:14 or a portion thereof.

In some embodiments, the invention is directed to a primer pair or a set or primer pairs for detecting any of PERV-A, PERV-B, PLHV-2, PCMV, PCV3, PLHV-1 and PLHV-3. In some embodiments, the primer pair comprises SEQ ID NOS: 1 and 2, SEQ ID NOS: 3 and 4, SEQ ID NOS: 5 and 6, SEQ ID NOS: 7 and 8, SEQ ID NOS: 9 and 10, SEQ ID NOS: 11 and 12, or SEQ ID NOS: 13 and 14.

In some embodiments, a forward and/or reverse oligonucleotide primer, primer pair or set of primer pairs, comprises one or more detectable labels to facilitate detection of the nucleic acid(s). In some embodiments, the label is a fluorescent label. In some embodiments, the label is a radiolabel. In some embodiments, the label is 6-carboxyfluorescein (6-FAM). In some embodiments, the label is tetramethylrhodamine (TAM RA). In some embodiments, the primer comprises both a 6-FAM (e.g., on the 5′ end) and TAMRA label (e.g., on the 3′ end).

In some embodiments, the PCMV nucleic acid is quantified using an oligonucleotide probe comprising SEQ ID NO:15 or a portion thereof.

In some embodiments, the PCV nucleic acid is quantified using an oligonucleotide probe comprising SEQ ID NO:16 or a portion thereof.

In some embodiments, the PLHV-1 nucleic acid is quantified using an oligonucleotide probe comprising SEQ ID NO:17 or a portion thereof.

In some embodiments, the PLHV-2 nucleic acid is quantified using an oligonucleotide probe comprising SEQ ID NO:18 or a portion thereof.

In some embodiments, the PLHV-3 nucleic acid is quantified using an oligonucleotide probe comprising SEQ ID NO:19 or a portion thereof.

In another embodiment, the invention provides a kit for detecting and quantifying the presence of porcine viruses in a sample from a xenotransplant subject, wherein the subject has received cells, tissues or an organ from a pig, comprising:
i) one or more sets of oligonucleotide primers comprising a forward and reverse primer for amplifying porcine virus nucleic acid in a sample using a PCR assay;
ii) one or more control templates;
iii) optionally one or more probes and/or dyes for detecting and quantifying the amplified porcine virus nucleic acid; and
iv) optionally one or more reagents for performing PCR, wherein the porcine virus comprises one or more of PERV-A, PERV-B, PCMV, PCV3, PLHV-1, PLHV-2, and PLHV-3.

In some embodiments, the kit comprises forward and reverse primers for amplifying nucleic acid from PERV-A, PERV-B, PCMV, PCV3, PLHV-1, PLHV-2, and PLHV-3.

In some embodiments, the kit further comprises instructions for conducting PCR, including generating a standard curve by conducting a PCR assay on the one or more control templates in order to calculate a copy number of porcine nucleic acid in a sample subjected to PCR using the one or more sets of oligonucleotide primers.

The kit comprises positive control templates, e.g., comprising the porcine virus nucleic acid, for quantitative detection. In some embodiments, positive control templates are porcine virus nucleic acid with concentration gradients, or can also include single samples of the templates that can be diluted in various ways (e.g., log scales) to be used in amplification reactions to make a standard curve.

In some embodiments, the positive control template can comprise samples (or be diluted) with a concentration of 1×10¹ copy/mL, a concentration 1×10² copy/mL, a concentration 1×10³ copy/mL, a concentration 1×10⁴ copy/mL, a concentration 1×10⁵ copy/mL, a concentration of 1×10⁶ copy/mL, and/or a concentration 1×10⁷ copy/mL.

In some embodiments, the kit comprises a fluorescent dye to bind PERV-A and PERV-B amplified nucleic acid.

In some embodiments, the kit comprises fluorescently labeled nucleic acid probes to detect amplified PCMV, PCV3, PLHV-1, PLHV-2, and PLHV-3 nucleic acid.

In some embodiments, the kit comprises forward and reverse oligonucleotide primers for amplifying PCMV nucleic acid, wherein the forward oligonucleotide primer comprises SEQ ID NO:1 or a portion thereof and the reverse oligonucleotide primer comprises SEQ ID NO:2 or a portion thereof.

In some embodiments, the kit comprises forward and reverse oligonucleotide primers for amplifying PCV nucleic acid, wherein the forward oligonucleotide primer comprises SEQ ID NO:3 or a portion thereof and the reverse oligonucleotide primer comprises SEQ ID NO:4 or a portion thereof.

In some embodiments, the kit comprises forward and reverse oligonucleotide primers for amplifying PLHV-1 nucleic acid, wherein the forward oligonucleotide primer comprises SEQ ID NO:5 or a portion thereof and the reverse oligonucleotide primer comprises SEQ ID NO:6 or a portion thereof.

In some embodiments, the kit comprises forward and reverse oligonucleotide primers for amplifying PLHV-2 nucleic acid, wherein the forward oligonucleotide primer comprises SEQ ID NO:7 or a portion thereof and the reverse oligonucleotide primer comprises SEQ ID NO:8 or a portion thereof.

In some embodiments, the kit comprises forward and reverse oligonucleotide primers for amplifying PLHV-3 nucleic acid, wherein the forward oligonucleotide primer comprises SEQ ID NO:9 or a portion thereof and the reverse oligonucleotide primer comprises SEQ ID NO:10 or a portion thereof.

In some embodiments, the kit comprises forward and reverse oligonucleotide primers for amplifying PERV-A nucleic acid, wherein the forward oligonucleotide primer comprises SEQ ID NO:11 or a portion thereof and the reverse oligonucleotide primer comprises SEQ ID NO:12 or a portion thereof.

In some embodiments, the kit comprises forward and reverse oligonucleotide primers for amplifying PERV-B nucleic acid, wherein the forward oligonucleotide primer comprises SEQ ID NO:13 or a portion thereof and the reverse oligonucleotide primer comprises SEQ ID NO:14 or a portion thereof.

In some embodiments, the kit comprises an oligonucleotide probe for detecting and quantifying PCMV, wherein the oligonucleotide probe comprises SEQ ID NO: 15 or a portion thereof.

In some embodiments, the kit comprises an oligonucleotide probe for detecting and quantifying PCV, wherein the oligonucleotide probe comprises SEQ ID NO: 16 or a portion thereof.

In some embodiments, the kit comprises an oligonucleotide probe for detecting and quantifying PLHV-1, wherein the oligonucleotide probe comprises SEQ ID NO: 17 or a portion thereof.

In some embodiments, the kit comprises an oligonucleotide probe for detecting and quantifying PLHV-2, wherein the oligonucleotide probe comprises SEQ ID NO: 18 or a portion thereof.

In some embodiments, the kit comprises an oligonucleotide probe for detecting and quantifying PLHV-3, wherein the oligonucleotide probe comprises SEQ ID NO: 19 or a portion thereof.

In some embodiments, the probe(s) comprises one or more detectable labels to facilitate detection of the nucleic acid(s). In some embodiments, the label is a fluorescent label. In some embodiments, the label is a radiolabel. In some embodiments, the label is 6-carboxyfluorescein (6-FAM). In some embodiments, the label is tetramethylrhodamine (TAMRA). In some embodiments, the probe(s) comprise both a 6-FAM (e.g., on the 5′ end) and TAMRA label (e.g., on the 3′ end).

While the invention has been described with reference to certain particular examples and embodiments herein, those skilled in the art will appreciate that various examples and embodiments can be combined for the purpose of complying with all relevant patent laws (e.g., methods described in specific examples can be used to describe particular aspects of the invention and its operation even though such are not explicitly set forth in reference thereto).

Other embodiments of the invention are discussed throughout this application. Any embodiment discussed with respect to one aspect applies to other aspects as well and vice versa. Each embodiment described herein is understood to be embodiments that are applicable to all aspects of the invention. It is contemplated that any embodiment discussed herein can be implemented with respect to any device, method, or composition, and vice versa. Furthermore, systems, compositions, and kits of the invention can be used to achieve methods of the invention.

### SAMPLE EMBODIMENTS

This section describes exemplary compositions and methods of the invention, presented without limitation, as a series of paragraphs, some or all of which may be alphanumerically designated for clarity and efficiency. Each of these paragraphs can be combined with one or more other paragraphs, and/or with disclosure from elsewhere in this application, including the materials incorporated by reference, in any suitable manner. Some of the paragraphs below expressly refer to and further limit other paragraphs, providing without limitation examples of some of the suitable combinations.

1. A method of detecting and quantifying the presence of porcine viruses in a sample from a xenotransplant subject, comprising,
i) providing a sample from the xenotransplant subject, wherein the subject has received cells, tissues or an organ from a pig;
ii) detecting the presence or absence of porcine virus nucleic acid in the sample that is amplified using a polymerase chain reaction (PCR) assay; and
iii) quantifying porcine virus nucleic acid detected in the sample, wherein the porcine virus comprises one or more of PERV-A, PERV-B, PCMV, PCV3, PLHV-1, PLHV-2, and PLHV-3.

2. The method of paragraph 1, wherein the cells, tissue or organ has been genetically modified.

3. The method of paragraph 1, wherein the cells, tissue or organ has been genetically modified to attenuate rejection by the subject.

4. The method of any of paragraphs 1-3, wherein the method comprises detecting the presence of absence of nucleic acid from PERV-A, PERV-B, PCMV, PCV3, PLHV-1, PLHV-2, and PLHV-3.

5. The method of any of paragraphs 1-4, wherein the amplified nucleic acid is quantified using a dye that binds to the porcine virus nucleic acid.

6. The method of paragraph 5, wherein the dye is a fluorescent dye.

7. The method of any of paragraphs 1-4, wherein the amplified nucleic acid is quantified using a labeled nucleic acid probe that binds to the nucleic acid.

8. The method of paragraph 7, wherein the probe is labeled with a fluorescent dye.

9. The method of any of paragraphs 1-8, wherein copy number of the porcine virus nucleic acid in the sample is quantified using a standard curve generated using amplified control nucleic acid.

10. The method of paragraph 9, wherein the amplified control nucleic acid is generated by polymerase chain reaction with a plasmid template comprising the porcine virus nucleic acid of a known quantity.

11. The method of any of paragraphs 1-10, wherein the subject is a human.

12. The method of paragraph 11, wherein the sample is selected from the group consisting of blood, plasma, and peripheral blood mononuclear cells (PBMCs).

13. The method of any of paragraphs 1-12, wherein the amplified PERV-A and PERV-B nucleic acid is quantified using a fluorescent dye that binds to the nucleic acid.

14. The method of any of paragraphs 1-13, wherein the amplified PCMV, PCV3, PLHV-1, PLHV-2, and PL HV-3 nucleic acid is quantified using a nucleic acid probe labeled with a fluorescent dye.

15. The method of any of paragraphs 1-14, wherein the lower limit of detection of the copy number of PERV-A, PERV-B and PLHV-2 is about 25 copies/μL.

16. The method of any of paragraphs 1-15, wherein the lower limit of detection of the copy number of PCMV, PCV3, PLHV-1 and PLHV-3 is about 10 copies/μL.

17. The method of any of paragraphs 1-16, wherein the number of cycles of PCR is thirty-six (36) or less.

18. The method of any of paragraphs 1-17, wherein PCMV nucleic acid is amplified using a forward oligonucleotide primer comprising SEQ ID NO:1 and a reverse oligonucleotide primer comprising SEQ ID NO:2.

19. The method of any of paragraphs 1-18, wherein PCV3 nucleic acid is amplified using a forward oligonucleotide primer comprising SEQ ID NO:3 and a reverse oligonucleotide primer comprising SEQ ID NO:4.

20. The method of any of paragraphs 1-19, wherein PLHV-1 nucleic acid is amplified using a forward oligonucleotide primer comprising SEQ ID NO:5 and a reverse oligonucleotide primer comprising SEQ ID NO:6.

21. The method of any of paragraphs 1-20, wherein PLHV-2 nucleic acid is amplified using a forward oligonucleotide primer comprising SEQ ID NO:7 and a reverse oligonucleotide primer comprising SEQ ID NO:8.

22. The method of any of paragraphs 1-21, wherein PLHV-3 nucleic acid is amplified using a forward oligonucleotide primer comprising SEQ ID NO:9 and a reverse oligonucleotide primer comprising SEQ ID NO:10.

23. The method of any of paragraphs 1-22, wherein PERV-A nucleic acid is amplified using a forward oligonucleotide primer comprising SEQ ID NO:11 and a reverse oligonucleotide primer comprising SEQ ID NO:12.

24. The method of any of paragraphs 1-23, wherein PERV-B nucleic acid is amplified using a forward oligonucleotide primer comprising SEQ ID NO:13 and a reverse oligonucleotide primer comprising SEQ ID NO:14.

25. The method of any of paragraphs 1-24, wherein PCMV nucleic acid is quantified using an oligonucleotide probe comprising SEQ ID NO:15.

26. The method of any of paragraphs 1-25, wherein PCV3 nucleic acid is quantified using an oligonucleotide probe comprising SEQ ID NO:16.

27. The method of any of paragraphs 1-26, wherein PLHV-1 nucleic acid is quantified using an oligonucleotide probe comprising SEQ ID NO:17.

28. The method of any of paragraphs 1-26, wherein PLHV-2 nucleic acid is quantified using an oligonucleotide probe comprising SEQ ID NO:18.

29. The method of any of paragraphs 1-26, wherein PLHV-3 nucleic acid is quantified using an oligonucleotide probe comprising SEQ ID NO:19.

30. The method of any of paragraphs 1-29, wherein the subject has received a genetically modified pig heart.

31. A kit for detecting and quantifying the presence of porcine viruses in a sample from a xenotransplant subject, wherein the subject has received cells, tissues or an organ from a pig, comprising:
i) one or more sets of oligonucleotide primers comprising a forward and reverse primer for amplifying porcine virus nucleic acid in a sample using a PCR assay;
ii) one or more control templates comprising the porcine virus nucleic acid;
iii) optionally one or more probes and/or dyes for detecting and quantifying the amplified porcine virus nucleic acid; and
iv) optionally one or more reagents for performing PCR,
wherein the porcine virus comprises one or more of PERV-A, PERV-B, PCMV, PCV3, PLHV-1, PLHV-2, and PLHV-3.

32. The kit of paragraph 31, wherein the kit comprises forward and reverse primers for amplifying nucleic acid from PERV-A, PERV-B, PCMV, PCV3, PLHV-1, PLHV-2, and PLHV-3.

33. The kit of paragraphs 31 or 32, wherein the dye binds to double stranded DNA and is a fluorescent dye.

34. The kit of any of paragraphs 31-33, wherein the probe is a labeled nucleic acid probe that binds to the porcine virus nucleic acid.

35. The kit of paragraph 34, wherein the probe is labeled with a fluorescent dye.

36. The kit of any of paragraphs 31-35, wherein the control template comprises porcine virus nucleic acid on a plasmid.

37. The kit of any of paragraphs 31-36, further comprising instructions for generating a standard curve by conducting a PCR assay on the one or more control templates in order to calculate a copy number of porcine nucleic acid in a sample subjected to PCR using the one or more sets of oligonucleotide primers.

38. The kit of any of paragraphs 31-37, wherein the kit comprises a fluorescent dye to bind PERV-A and PERV-B amplified nucleic acid.

39. The kit of any of paragraphs 31-38, wherein the kit comprises fluorescently labeled nucleic acid probes to detect amplified PCMV, PCV3, PLHV-1, PLHV-2, and PLHV-3 nucleic acid.

40. The kit of any of paragraphs 31-39, wherein the kit comprises forward and reverse oligonucleotide primers for amplifying PCMV nucleic acid, wherein the forward oligonucleotide primer comprises SEQ ID NO:1 and the reverse oligonucleotide primer comprises SEQ ID NO:2.

41. The kit of any of paragraphs 31-40, wherein the kit comprises forward and reverse oligonucleotide primers for amplifying PCV3 nucleic acid, wherein the forward oligonucleotide primer comprises SEQ ID NO:3 and the reverse oligonucleotide primer comprises SEQ ID NO:4.

42. The kit of any of paragraphs 31-41, wherein the kit comprises forward and reverse oligonucleotide primers for amplifying PLHV-1 nucleic acid, wherein the forward oligonucleotide primer comprises SEQ ID NO:5 and the reverse oligonucleotide primer comprises SEQ ID NO:6.

43. The kit of any of paragraphs 31-42, wherein the kit comprises forward and reverse oligonucleotide primers for amplifying PLHV-2 nucleic acid, wherein the forward oligonucleotide primer comprises SEQ ID NO:7 and the reverse oligonucleotide primer comprises SEQ ID NO:8.

44. The kit of any of paragraphs 31-43, wherein the kit comprises forward and reverse oligonucleotide primers for amplifying PLHV-3 nucleic acid, wherein the forward oligonucleotide primer comprises SEQ ID NO:9 and the reverse oligonucleotide primer comprises SEQ ID NO:10.

45. The kit of any of paragraphs 31-44, wherein the kit comprises forward and reverse oligonucleotide primers for amplifying PERV-A nucleic acid, wherein the forward oligonucleotide primer comprises SEQ ID NO:11 and the reverse oligonucleotide primer comprises SEQ ID NO:12.

46. The kit of any of paragraphs 31-45, wherein the kit comprises forward and reverse oligonucleotide primers for amplifying PERV-B nucleic acid, wherein the forward oligonucleotide primer comprises SEQ ID NO:13 and the reverse oligonucleotide primer comprises SEQ ID NO:14.

47. The kit of any of paragraphs 31-46, wherein the kit comprises an oligonucleotide probe for detecting and quantifying PCMV, wherein the oligonucleotide probe comprises SEQ ID NO:15.

48. The kit of any of paragraphs 31-47, wherein the kit comprises an oligonucleotide probe for detecting and quantifying PCV3, wherein the oligonucleotide probe comprises SEQ ID NO:16.

49. The kit of any of paragraphs 31-47, wherein the kit comprises an oligonucleotide probe for detecting and quantifying PLHV-1, wherein the oligonucleotide probe comprises SEQ ID NO:17.

50. The kit of any of paragraphs 31-47, wherein the kit comprises an oligonucleotide probe for detecting and quantifying PLHV-2, wherein the oligonucleotide probe comprises SEQ ID NO:18.

51. The kit of any of paragraphs 31-47, wherein the kit comprises an oligonucleotide probe for detecting and quantifying PLHV-3, wherein the oligonucleotide probe comprises SEQ ID NO:19.

Aspects of the present teachings may be further understood in light of the following examples, which should not be construed as limiting the scope of the present teachings in any way.

### EXAMPLES

### Example 1. Quantitative PCR Assays for Detection of Porcine Viral RNAs after Xenotransplant

### 2. Materials and Methods

### 2.1. Human and Donor Pig Specimens

Blood specimens from two cardiac-xenotransplant recipients were collected at University of Maryland School of Medicine, Baltimore following IRB protocol. Blood samples were collected at 7-days and 35 days after xenotransplants. PBMCs and plasma were separated following standard protocols at the Institute of Human Virology, University of Maryland School of Medicine, Baltimore. The genetically engineered donor pig as described earlier by Eyestone et al., was received from a biosecure Revivicor facility in Blacksburg, Virginia (Eyestone et al., Cham, Switzerland: Springer, (2020), 121-40). Porcine tissue samples were collected at the hospital-adjacent animal facility of University of Maryland School of Medicine, Baltimore.

2.2. Isolation of Total RNA and Preparation of cDNA

Total RNA was isolated from all pig donor tissues, human PBM C and plasma samples using RNeasy Mini Kit (Qiagen, USA) and Direct-zol™ RNA Miniprep (ZYMO, USA), respectively, following the manufacturer's protocol. RNA quality and quantity were evaluated using a Nanodrop ND-1000 Spectrophotometer (Thermo Fisher Scientific, USA). cDNAs were synthesized using Transcriptor High Fidelity CDNA Synthesis Kit (Roche, USA). In the case of tissues and PBMCs, cDNAs were prepared from 200 ng total RNA in 20 μl volume, and 2 μl of that cDNA is used for real-time qPCR, which is equivalent to cDNA prepared from 20 ng. Thus, the detected copy numbers from standard curves were divided by a factor of 20 ((200/20)×2) to present the viral copy numbers in tissues or PBM C samples using a unit of copies/ng RNA. In the case of plasma samples, total RNA was isolated from 500 μl of plasma samples and eluted in 40 μl of RNase free water. 10 μl of that extracted total RNA (equivalent to 500 μl/40×10=125 μl of plasma) was used to synthesize cDNA at a final volume of 20 μl and 2 μl of that cDNA is used for real-time qPCR. Thus, the detected copy numbers from standard curves were divided by a factor of 12.5((125/20)×2) to present the viral copy numbers in plasma specimens using a unit of copies/μl of plasma.

2.3. Quantitative Real-Time qPCR Assay

Quantitative real-time qPCR assays were performed in a the QuanStudio™ 3 System (Thermo Fisher Scientific, USA) using absolute quantification method. The copy numbers of PERV-A and PERV-B were evaluated using PowerUp™ SYBR™ Green Master M ix (Thermo Fisher Scientific, USA) and the copy numbers of PCMV, PCV3, and PLHV-1, PLHV-2, and PLHV-3 were evaluated using TaqM an Universal PCR master Mix (Thermo Fisher Scientific, USA). PCR amplifications were performed using the following conditions: for SY BR Green chemistry, initial denaturation at 95° C. for 2 min followed by 40 cycles of amplification with denaturation at 95° C. for 15 s, annealing and extension at 60° C. for 1 min; for TaqM an chemistry, initial denaturation at 95° C. for 10 min followed by 40 cycles of amplification with denaturation at 95° C. for 15s, annealing and extension at 60° C. for 1 min.

### 2.4. Plasmid Cloning

In the case of PCV3, PLHV-1, PLHV-2, and PLHV-3 we have generated plasmid clones containing the section of the viral RNAs to be amplified by the respective primer sets. We have used annealing oligonucleotides method to generate small stretches of blunt-ended double stranded DNA products for the respective section of the viral RNAs. The sequences of the top and bottom strand oligos with accession numbers of the mRNA sequences for all the selected viruses are presented in Table 1.

TABLE 1
The sequences of the top and bottom strand oligos used in annealing
oligonucleotides method to generate small stretch of blunt-ended double stranded
DNA.

| Target | | Sequence 5′-3′ |
|---|---|---|
| PCV3 | Fwd | CGGACTTGTAACGAATCCAAACTTCTTTCGTGCCGTAGAAGTCTGTCATTCCAGTTTTTTCCGGGACATAAATGCTCC |
| | | SEQ ID NO: 20 |
| | Rev | GGAGCATTTATGTCCCGGAAAAAACTGGAATGACAGACTTCTACGGCACGAAAGAAGTTTGGATTCGTTACAAGTCCG |
| | | SEQ ID NO: 21 |
| PLHV-1 | Fwd | ACAGCGACCTGGTCTACTGAATCGCCGCTAACAGGTCACTATGGAACACACGATTCAAGCC SEQ ID NO: 22 |
| | Rev | GGCTTGAATCGTGTGTTCCATAGTGACCTGTTAGCGGCGATTCAGTAGACCAGGTCGCTGT SEQ ID NO: 23 |
| PLHV-2 | Fwd | GCTGCCAATAGGTCAATATGGAACATACGATTCAAGCCACGGTGAAAGAGCAACCAGCGAAA SEQ ID NO: 24 |
| | Rev | TTTCGCTGGTTGCTCTTTCACCGTGGCTTGAATCGTATGTTCCATATTGACCTATTGGCAGC SEQ ID NO: 25 |
| PLHV-3 | Fwd | AACAGCGCCAGAAAAAAAGGACCCCAAAGAGGAAAATCAATTTTATGGTTCACCTTCTACCTTTCC SEQ ID NO: 26 |
| | Rev | GGAAAGGTAGAAGGTGAACCATAAAATTGATTTTCCTCTTTGGGGTCCTTTTTTTCTGGCGCTGTT SEQ ID NO: 27 |

Respective oligos were reconstituted to a stock concentration of 100 μM in specific annealing buffer (10 mM Tris pH 7.5, 1 mM EDTA and 50 mM NaCl). 10 μM top and bottom strand oligos were mixed and kept in a heat block at 95° C. for 15 min followed by gradual cooling to room temperature in the heat block for 45 min to allow annealing of the oligos. Then the blunt-ended annealed oligos products were directly inserted into a pCR-Blunt II-TOPO vector using Zero Blunt™ TOPO PCR Cloning Kit (Invitrogen, USA). For PCMV we have amplified PCR product with cDNA prepared from PCMV infected PT-K 75 cells as template and using PCMV-specific primers. Then, the PCR product was cloned in TOPO vector as mentioned above. Positive plasmid clones were selected and extracted by QIA prep® Spin Miniprep Kit (Qiagen, USA) and resuspended in TE buffer. The first batch of extracted purified plasmids served as standards for PCMV, PCV3, PLHV-1, PLHV-2, and PLHV-3.

### 2.5. Cell Lines and Positive Controls Used for PCR

Porcine embryonic kidney cell line PK-15 (CCL-33) and PT-K 75 cell line (ATC CRL-2528TM) of porcine (Sus scrofa) nasal turbinate origin were purchased form ATCC and cultured in Dulbecco's Modified Eagle's Medium (DMEM) supplemented with 10% FBS and 1% Penicillin-Streptomycin at 37° C. in a 5% CO2 atmosphere. PK-15 cell line contains integrated DNA for PERV-A and PERV-B. (ref) The genomic DNA isolated from PK-15 cell line using Quick-DNA miniprep kit (Zymo Research) was used as standards and positive control for PERV-A and PERV-B PCR amplification. For PCMV, kanitz strain of PCMV (ATCC VR-1499TM) was purchased form ATCC. When PT-K 75 cells reached 70% confluency, PCMV virus were added to infect the cells. After 1 hr of incubation cells were washed 3 times with PBS (pH 7.4) to remove free viruses from the culture. Infected cells were then cultured in fresh media for 72 hr. Then, total RNA was isolated, and cDNA were prepared as described in section 2.2. The cDNA was used as positive control for PCMV PCR amplification. In the case of PCV3, PLHV-1, PLHV-2, and PLHV-3 we have used extracted plasmid clones (as described in section 2.4) for each of the viral isolates as positive control. Plasmid used for positive control were extracted in separate batches than the batch of standards.

### 2.6. Primer Sets for PCR Amplification of Porcine Viruses PCMV, PCV3, PLHV-1, PLHV-2, PLHV-3, PERV-A and PERV-B

Primers and probe sequences for PCMV polymerase gene were described previously by Mueller et al. (Mueller et al., J Virol, (2002), 76:4734-40). Primers and probe sequences for PCV3 capsid gene were adopted from previously published manuscript by Wang et al., where it was confirmed that the primers were specific for PCV3 but not for PCV1 and PCV2 (Wang et al., J Virol Methods, (2017), 248:177-80). Primers and probe sequences for PLHV-1, PLHV-2 and PLHV-3 glycoprotein B (gB) genes were described previously by McMahon et al. (McMahon et al., Vet Microbiol, (2006), 116:60-8). All probes were designed with 6-FAM fluorophore label at 5′-end and TAM RA quencher at the 3′-end. Primer sequences for envelop genes for PERV-A and PERV-B were adopted from previously published manuscripts by Bösch et al. and Mazurek et al. (Bosch et al., J Virol, (2000), 74:8575-81; Mazurek et al., Curr Microbiol, (2013), 67:505-14). The primer-probe sequences were presented in Table 2.

TABLE 2
The primer-probe sequences used in the study.

| | | | | Amplicon | |
|---|---|---|---|---|---|
| PCR | | Primer/ | | (Base | Accession |
| Assay | Target | Probe | Sequence 5′-3′ | pair) | Number |
| TaqMan | PCMV | PCMV-Fwd | GTT CTG GGA TTC CGA GGT TG SEQ ID NO: 1 | 63 | AF268039.2 |
| | (poly- | PCMV-Rev | ACT TCG TCG CAG CTC ATC TGA SEQ ID NO: 2 | | |
| | merase) | PCMV-probe | 6FAM-CAG GGC GGC GGT CGA GCT C-TAMRA SEQ | | |
| | | | ID NO: 15 | | |
| | PCV3 | PCV3-Fwd | CGG ACT TGT AAC GAA TCC AAA CT SEQ ID NO: 3 | 78 | KX778720.1 |
| | (capsid) | PCV3-Rev | GGA GCA TTT ATG TCC CGG AAA SEQ ID NO: 4 | | |
| | | PCV3-probe | 6FAM-CTT TCG TGC CGT AGA AGT CTG TCA TTC | | |
| | | | CA-TAMRA SEQ ID NO: 16 | | |
| | PLHV-1 | PLHV1-Fwd | ACA GCG ACC TGG TCT ACT GAA TC SEQ ID NO: 5 | 61 | AF478169.1 |
| | (glyco- | PLHV1-Rev | GGC TTG AAT CGT GTG TTC CA SEQ ID NO: 6 | | |
| | protein B) | PLHV1-probe | 6-FAM-CCG CTA ACA GGT CACT-TAMRA SEQ ID | | |
| | | | NO: 17 | | |
| | PLHV-2 | PLHV2-Fwd | GCT GCC AAT AGG TCA ATA TGG AA SEQ ID NO: 7 | 62 | AY170317.1 |
| | (glyco- | PLHV2-Rev | TTT CGC TGG TTG CTC TTT CA SEQ ID NO: 8 | | |
| | protein B) | PLHV2-probe | 6FAM-CAT ACG ATT CAA GCC AC-TAMRA SEQ ID | | |
| | | | NO: 18 | | |
| | PLHV-3 | PLHV3-Fwd | AAC AGC GCC AGA AAA AAA GG SEQ ID NO: 9 | 66 | AY170316.1 |
| | (glyco- | PLHV3-Rev | GGA AAG GTA GAA GGT GAA CCA TAA AA SEQ ID | | |
| | protein B) | | NO: 10 | | |
| | | PLHV3-probe | 6FAM-CCA AAG AGG AAA ATC-MGB SEQ ID NO: 19 | | |
| SYBR | PERV | PERV-A-Fwd | GAGATGGAAAGATTGGCAACAGCG SEQ ID NO: 11 | 364 | AY099323.1 |
| Green | (envelop) | PERV-A-Rev | AGTGATGTTAGGCTCAGTGGGGAC SEQ ID NO: 12 | | |
| | PERV | PERV-B-Fwd | AATTCTCCTTTGTCAATTCCGGCCC SEQ ID NO: 13 | 270 | AY099324.1 |
| | (envelop) | PERV-B-Rev | CCAGTACTTTATCGGGTCCCACTG SEQ ID NO: 14 | | |

### 2.7. In Silico Alignment Analysis of Primers/Probes for Specificity and Cross-reactivity

To Verify the Specificity of the Primers and Probes to Target Viral Sequences and possible cross-reactivity with non-target sequences of porcine or human origin, nucleotide BLAST analysis was performed using the National Center for Biotechnology Information (NCBI) website https://blast.ncbi.nlm.nih.gov/Blast.cgi. Sequences of full-length amplicons between the forward and reverse primers were used as query in the ‘blastn’ analysis applying ‘Somewhat similar sequence’ algorithm. We have excluded matching hits from the blast results if the matching sequences correspond to predicted sequences, clones or chromosomal DNA sequences. Sequence mismatches in the primer and probe sequences were analyzed from sequence alignments using ‘MSA viewer’ tab in the result pages.

### 2.8. Statistics

Statistical analysis was performed using GraphPad Prism version 9.2.0 (GraphPad Software, Inc., San Diego, CA, United States). Pearson's correlations were performed to evaluate the linearity in the PCR amplifications.

### 3. Results

### 3.1. Specificity and Cross-Reactivity of the Primer/Probe Sequences

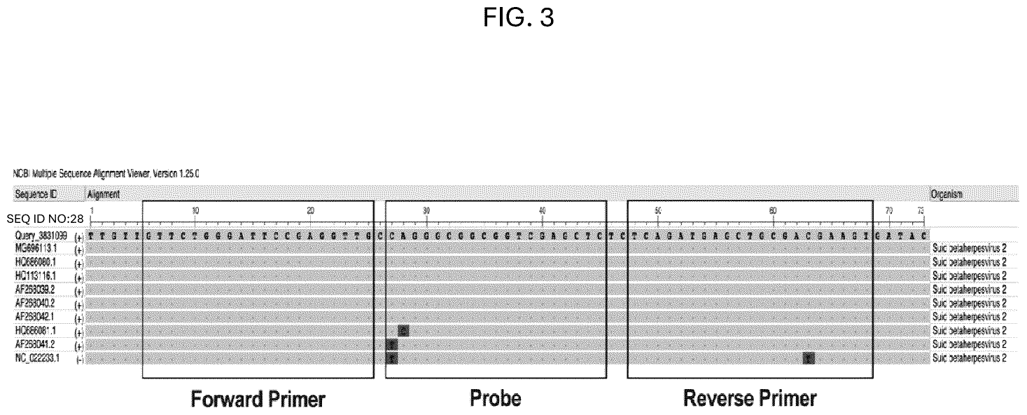

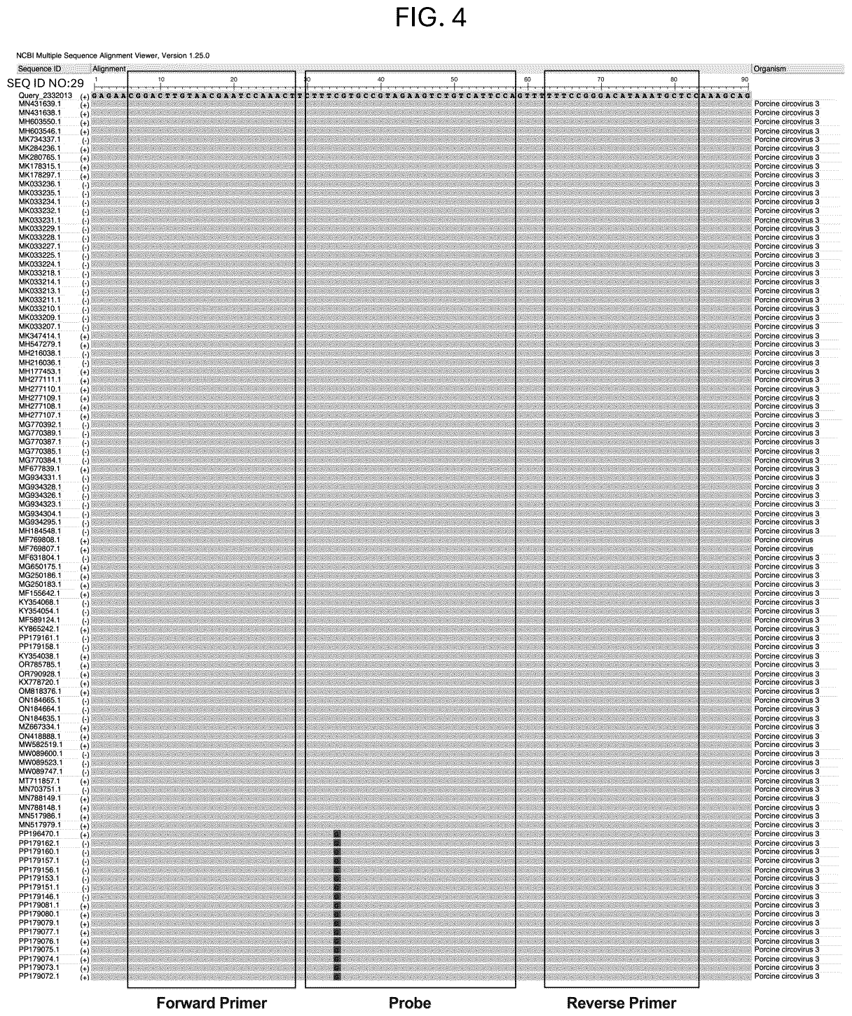

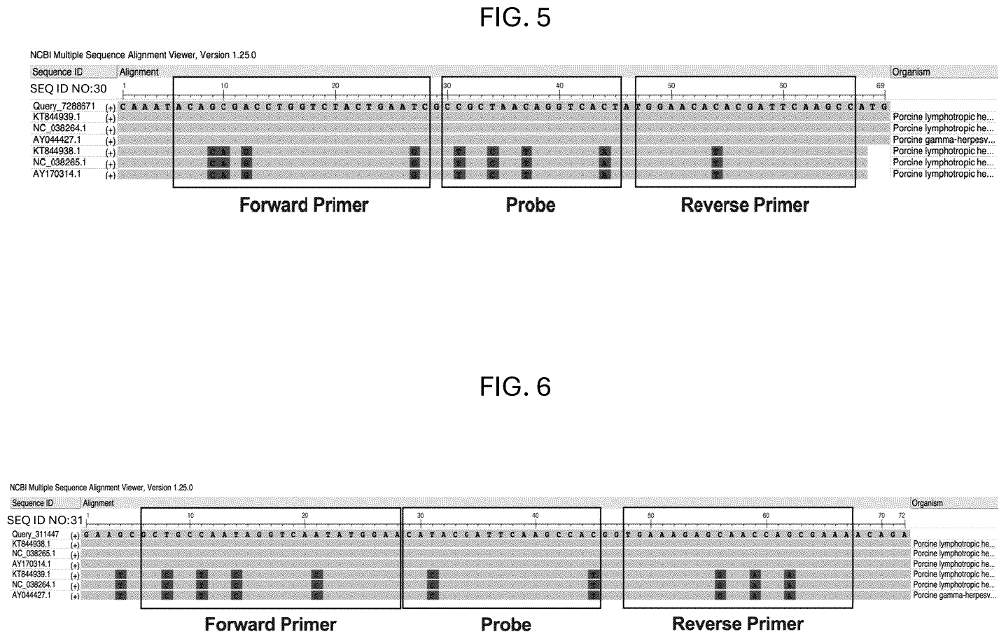

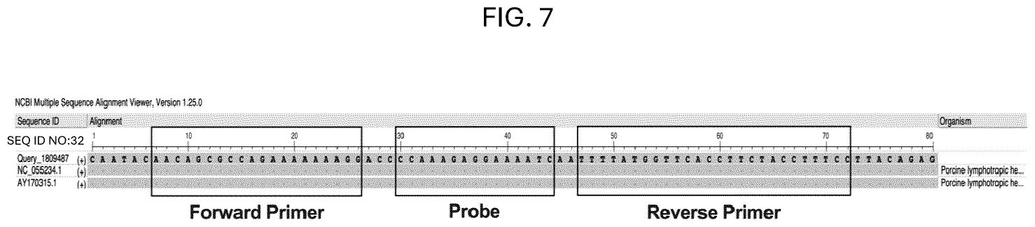

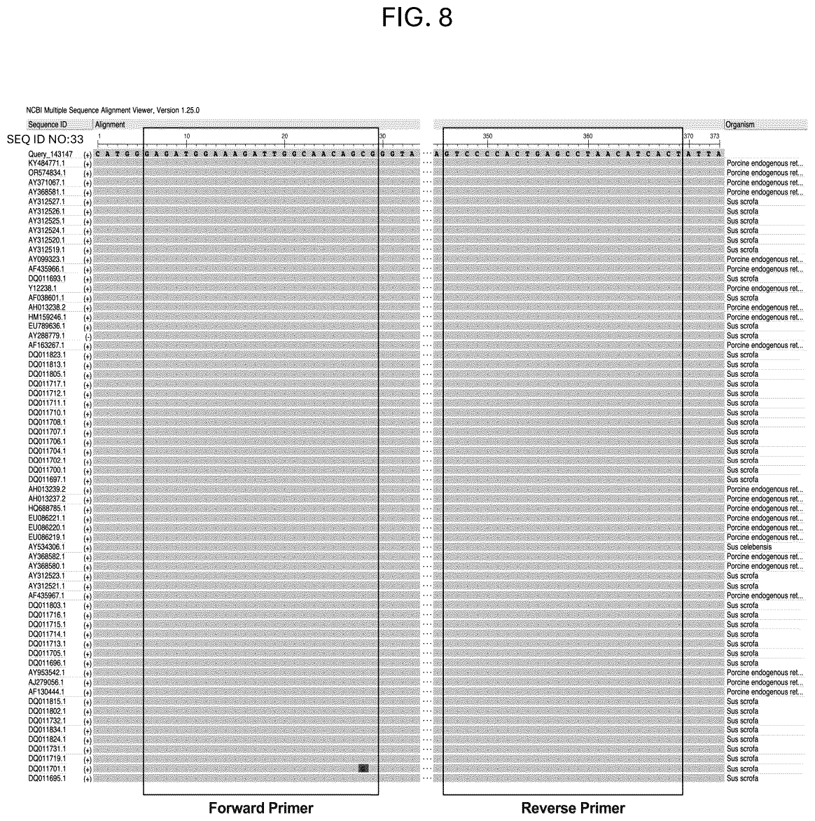

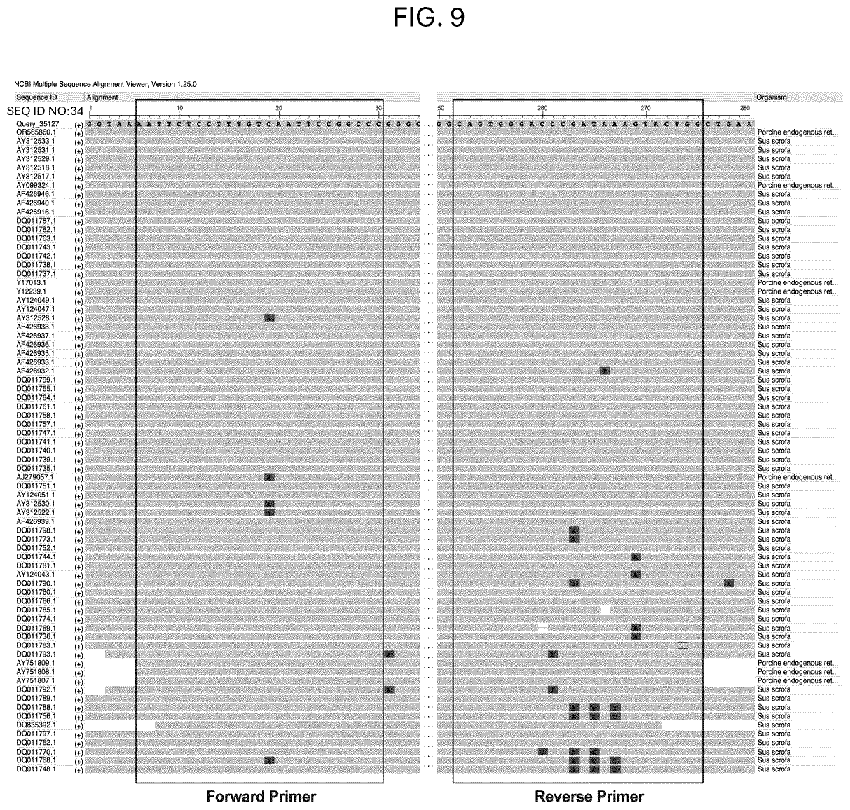

The specificity of the primer/probe sequences used to amplify porcine nucleic acids in this study were tested in pig samples earlier by different study groups. However, to further evaluate the specificity of the primer/probe sequences we performed in silico sequence alignment analysis to verify the specificity of the primers and probes across multiple published sequences for each porcine virus using NCBI blast analysis (FIGS. 3-9). Using the query sequence of PCMV (accession no AF268039.2) we found 9 hits, all for PCMV (also known as Suid betaherpesvirus 22). Six of them had complete sequence match with primers/probe, while two had single mismatch in probe (HQ686081.1 and A F268041.2) and one had two mismatches, one in probe and one in reverse primer (NC_022233.1) (FIG. 3). For PCV3, we found 100 matching hits, all for PCV3 sequence. 83 of them had complete match with primers/probe and 17 of them had only 1 mismatch in the probe sequence (FIG. 4). This finding corroborated with the previous report by Wang et al. where the authors found that PCV3 probe had only a single mismatch among 3 of the 13 PCV3 sequences they had tested (Wang et al., J Virol Methods, (2017), 248:177-80). PLHV-1 and PLHV-2 both had the same 6 hits in the blast analysis, three of them were for PLHV-1 and the rest three were for PL HV-2 (FIGS. 5,6). Primers and probe targeting PLHV-1 matched completely with the PLHV-1 sequences but had 4 mismatches in forward primer, 4 mismatches in probe and 1 mismatch in reverse primer with the sequences of PLHV-2 (FIG. 5). Similarly, primers and probe targeting PLHV-2 matched completely with the PLHV-2 sequences but had 5 mismatches in forward primer, 2 mismatches in probe and 3 mismatches in reverse primer with the sequences of PLHV-1 (FIG. 6). PLHV-3 had only 2 hits, both correspond to PLHV-3 sequences with complete match with primers and probe (FIG. 7). Query sequence for PERV-A had 65 hits with complete match of the primer sequences in 64 hit and only 1 mismatch in forward primer in 1 hit (DQ011701.1) (FIG. 8). Sequences of 59 of the 65 hits correspond to PERV-A. Among the remaining 6 hits, one corresponds to PERV-A/B recombinant sequence (DQ011834.1), two hits correspond to PERV-A/C recombinant sequences (AY 534306.1 and A Y 953542.1), and three hits correspond to PERV-C (OR574834.1, AF130444.1, HM 159246.1) as described in the NCBI database. However, upon careful comparison of the amino acid sequences of these hits with previously published amino acid sequences of PERV-A and PERV-C we confirmed all three PERV-C hits actually represent PERV-A sequence (Martin et al., Virol J, (2006), 3:91). Finally, for PERV-B query sequence we found 73 hits, all of which correspond to PERV-B sequences. 59 of the sequence hits had complete matches with both forward and reverse primers, 4 hits had single mismatch in forward primer, 10 hits had single mismatch in reverse primer, 4 hits had 3 mismatches in reverse primer, and only 1 hit had 1 mismatch in forward primer along with 3 mismatches in reverse primer (FIG. 9).

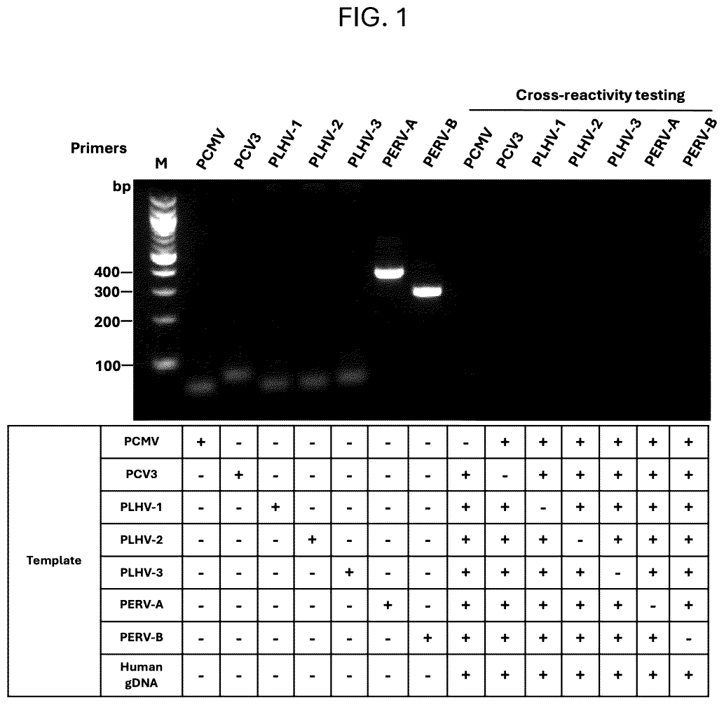

Since the in silico analyses suggested very high specificity of the primers/probe sets used in our assays with the corresponding target porcine viruses, we tested their specificity by semiquantitative PCR and gel electrophoresis. All the primer sets successfully amplified the positive controls in semi-quantitative PCR (lanes 2-8 of FIG. 1). Moreover, to determine the possible cross-reactivity of each virus-specific primer sets, semi-quantitative PCR were performed using a mix of positive controls of all other viruses and human gDNA from healthy volunteer PBMCs. A concentration of 10⁵ copies/ul of positive controls were used for the testing of cross-reactivity. As shown in FIG. 1 (lanes 9-15), none of the primer sets amplified any PCR product in 35 cycles of amplification from positive controls of different viruses or human gDNA. These data confirmed the specificity of the primer sequences used in our real-time qPCR assays.

3.2. Preparation of Standards of Known Copy Numbers for the Standard Curve Analysis in Absolute Quantification Using Real-Time qPCR

Standard curves generated by standards of known copy numbers are an essential component of absolute quantification method in real-time qPCR for accurate determination of viral RNA copy numbers in human samples. Since it is difficult to find positive controls for PCMV, PCV3, PLHV-1, PLHV-2, and PLHV-3, we employed annealing oligonucleotides method to generate small stretch of blunt-ended double stranded DNA products harboring the respective viral DNA sequences and cloned them in plasmid vectors. Then the plasmids were extracted and copy numbers were calculated using the following formula as previously described by Lee et al. (Lee et al., J Biotechnol, (2006), 123:273-80).

$$For PCMV , PCV 3 , PLHV - 1 , PLHV - 2 and PLHV - 3 ,$$

$$Copies / μL = [ plasmid DNA concentration ( g / μL ) \times 6.02 \times {10}^{23} ( copies / mol ) ] / ( plasmid length ( bp ) \times 660 ( g / mol / bp ) )$$

$$For PERV - A and PERV - B ,$$

$$Copies / μL = [ PK - 15 gDNA concentration ( g / μL ) \times 6.02 \times {10}^{23} ( copies / mol ) ] / ( PK - 15 gDNA length ( bp ) \times 660 ( g / mol / bp ) \times 46 )$$

The genomic DNA (gDNA) isolated from PK-15 cell line was used as standards for PERV-A and PERV-B. For copy number calculation in case of PERVs a factor of 46 is introduced as each PK-15 cells contains 46 copies of PERV (Fiebig et al. 2018). Stocks of the standards were prepared with a concentration of 10⁸ copies/μL for the plasmids and 10⁶ copies/μL for PK-15 gDNA. Ten-fold dilutions of the stocks ranging from 10 to 10⁸ copies/μL for plasmids and 10 to 10⁶ copies/μL for PK-15 gDNA were used to generate standard curves in real-time qPCR (FIG. 4A-G).

### 3.3. Analysis of the Lower Limit of Detection (LLoD) of the Assay

The LLoDs for each assay were determined by testing multiple replicates of low concentration positive controls ranging from 1-100 copies/μL. The real-time qPCRs were performed in 2-3 replicates for each primer/probe sets on three different days. As indicated in Table 3, the detection rate drops below 100% at concentrations below 25 copies/μL for PERV-A, PERV-B and PLHV-2, while for PCMV, PCV3, PLHV-1 and PLHV-3 the detection rates were 100% at and above 10 copies/μL. In all the assays we observed that the detection rates drop below 100% when the Ct values in real-time qPCR crosses 36 cycles, indicating PCR amplification beyond 36 cycle could be unreliable. With this data we claim 25 copies/μL as a reliable LLOD for PERV-A, PERV-B and PLHV2 and 10 copies/μL as a reliable LLOD for PCMV, PCV3, PL HV 1 and PLHV2 in our real-time qPCR assays.

TABLE 3
Lower limit of detection (LLoD) determined by low concentration
positive controls for each porcine virus.

| | | Number of | Number of | |
|---|---|---|---|---|
| Target | Concentrations | Replicates | Replicates | Detection |
| Virus | (Copies/ul) | tested | detected | Rate (%) |
| PERV-A | 100 | 9 | 9 | 100 |
| | 50 | 9 | 9 | 100 |
| | 25 | 9 | 9 | 100 |
| | 10 | 9 | 6 | 66.7 |
| | 5 | 9 | 3 | 33.3 |
| | 2.5 | 9 | 2 | 22.2 |
| | 1 | 9 | 1 | 11.1 |
| PERV-B | 100 | 9 | 9 | 100 |
| | 50 | 9 | 9 | 100 |
| | 25 | 8 | 8 | 100 |
| | 10 | 9 | 6 | 66.7 |
| | 5 | 9 | 2 | 22.2 |
| | 2.5 | 9 | 2 | 22.2 |
| | 1 | 9 | 1 | 11.1 |
| PCMV | 100 | 9 | 9 | 100 |
| | 50 | 9 | 9 | 100 |
| | 25 | 9 | 9 | 100 |
| | 10 | 9 | 9 | 100 |
| | 5 | 9 | 9 | 100 |
| | 2.5 | 9 | 7 | 77.8 |
| | 1 | 9 | 1 | 11.1 |
| PCV3 | 100 | 9 | 9 | 100 |
| | 50 | 9 | 9 | 100 |
| | 25 | 9 | 9 | 100 |
| | 10 | 9 | 9 | 100 |
| | 5 | 9 | 9 | 100 |
| | 2.5 | 9 | 8 | 88.9 |
| | 1 | 9 | 6 | 66.7 |
| PLHV-1 | 100 | 9 | 9 | 100 |
| | 50 | 8 | 8 | 100 |
| | 25 | 9 | 9 | 100 |
| | 10 | 9 | 9 | 100 |
| | 5 | 9 | 7 | 77.8 |
| | 2.5 | 9 | 2 | 22.2 |
| | 1 | 9 | 0 | 0 |
| PLHV-2 | 100 | 9 | 9 | 100 |
| | 50 | 8 | 8 | 100 |
| | 25 | 9 | 9 | 100 |
| | 10 | 9 | 5 | 55.6 |
| | 5 | 9 | 1 | 11.1 |
| | 2.5 | 9 | 0 | 0 |
| | 1 | 9 | 0 | 0 |
| PLHV-3 | 100 | 9 | 9 | 100 |
| | 50 | 8 | 8 | 100 |
| | 25 | 9 | 9 | 100 |
| | 10 | 9 | 9 | 100 |
| | 5 | 9 | 9 | 100 |
| | 2.5 | 9 | 9 | 100 |
| | 1 | 9 | 6 | 66.7 |

Using these LLoDs from the assays the effective LLOD in human PBM C specimens (as described in method section 2.2) will be 1.25 copies/ng RNA for PERV-A, PERV-B and PLHV-2 and 0.5 copies/ng RNA for PCMV, PCV3, PLHV-1 and PLHV-3. Similarly, the effective LLOD in human plasma specimens will be 2 copies/μl of plasma for PERV-A, PERV-B and PLHV2 and 0.8 copies/μl of plasma for PCMV, PCV3, PLHV1 and PLHV2.

### 3.4. Assessment of Linearity

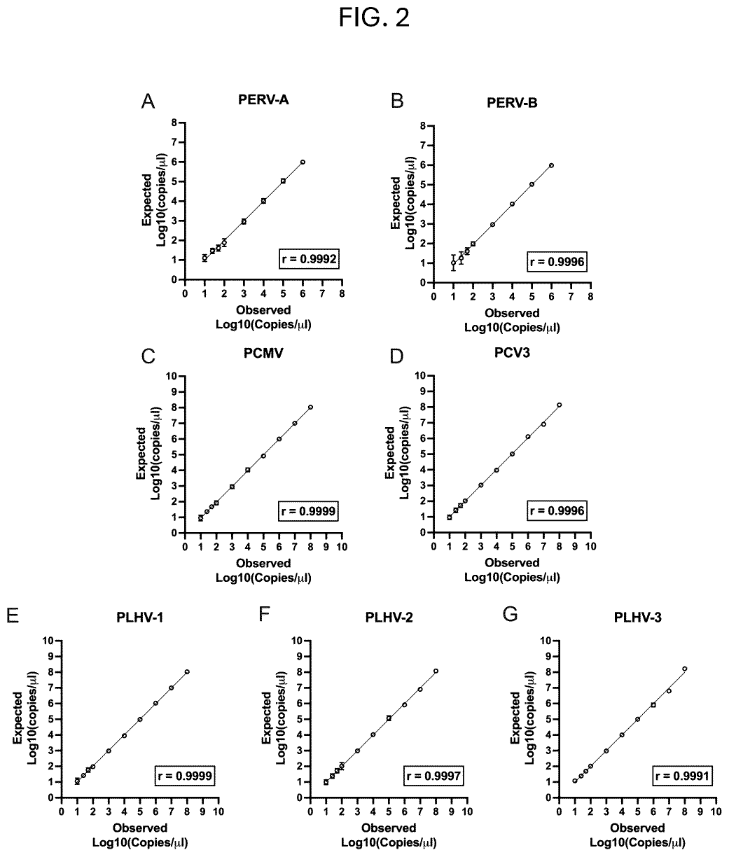

To assess the linearity across a long rage of concentrations we have tested the performances of all the assays with a series of dilutions of the positive controls. For PERV-A and PERV-B, 8 panels of different concentrations ranging from 10-1,000,000 copies/μL (1.0 Log copies/μL to 6.0 Log copies/μL) were verified using PK-15-gDNA, and for PCMV, PCV3, PLHV-1, PLHV-2 and PLHV-3, 10 panels of different concentrations ranging from 10-100,000,000 copies/μL (1.0 Log copies/μL to 8.0 Log copies/μL) were verified using the plasmid positive controls. 3 replicates for each concentration were used for amplifications and the real-time qPCR assays were performed on three different days resulting in total 9 replicates for each concentration. As shown in FIGS. 2A-G, the Pearson's correlation coefficients (r) between expected concentrations and the mean observed concentrations determined by the standard curves were above 0.999 in all the assays indicating strong linearity within the concentration range 10-1,000,000 copies/μL for PERV-A and PERV-B, and 10-100,000,000 copies/μL for PCMV, PCV3, PLHV-1, PLHV-2 and PLHV-3. Based on these data we claim that the upper limit of quantitations (ULoQ) in our real-time qPCR assays are 6.0 Log copies/μL to detect PERV-A and PERV-B and 8.0 Log copies/μL for PCMV, PCV3, PLHV-1, PLHV-2 and PLHV-3.

3.5. Determination of Copy Numbers of all Viral RNAs in Donor Pig Tissues and Human Samples Using Established Real-Time qPCR Assays

Finally, we tested our real-time qPCR-based assays to determine the RNA copy numbers of porcine viruses in heart, liver, spleen, lung, lymph node, PB M C and plasma of a donor pig, and human PBMC and plasma specimens from two xenotransplant recipients. Each specimen was tested in 3 replicates and copy numbers were calculated with mean Ct values using the standard curves as described in section 2.2. In the pig donor tissues PERV-A and PERV-B RNAs were detected in all organs tested, while RNAs for rest of the viruses were either undetected or below LLOD (Table 4). However, all the human specimens were negative for porcine viral RNA until 1 month after the cardiac xenotransplant (Table 5).

TABLE 4
viral RNA copy numbers in porcine tissue specimens.

| | | PERV-A | | PERV-B | | PCMV | | PCV3 | | PLHV-1 | | PLHV-2 | | PLHV-3 | |
|---|---|---|---|---|---|---|---|---|---|---|---|---|---|---|---|
| | | | Copy | | Copy | | Copy | | Copy | | Copy | | Copy | | Copy |
| | | | Number | | Number | | Number | | Number | | Number | | Number | | Number |
| | | | (Copies/ | | (Copies/ | | (Copies/ | | (Copies/ | | (Copies/ | | (Copies/ | | (Copies/ |
| | | | ng of | | ng of | | ng of | | ng of | | ng of | | ng of | | ng of |
| Samples | | Ct | RNA) | Ct | RNA) | Ct | RNA) | Ct | RNA) | Ct | RNA) | Ct | RNA) | Ct | RNA) |
| Pig | Heart | 29.5 | 24.96 | 30 | 12.42 | 37.2 | Below | 36.1 | Below | 38.5 | Below | NA | UD | 39 | Below |
| donor | | | | | | | LLoD | | LLoD | | LLoD | | | | LLoD |
| tissues | Liver | 29.2 | 29.87 | 30 | 11.99 | 36.1 | Below | 38.2 | Below | 37.3 | Below | NA | UD | NA | UD |
| | | | | | | | LLoD | | LLoD | | LLoD | | | | |
| | Spleen | 29.3 | 26.78 | 30 | 12.39 | 36.3 | Below | 37 | Below | 37.9 | Below | NA | UD | NA | UD |
| | | | | | | | LLoD | | LLoD | | LLoD | | | | |
| | Lung | 27.8 | 72.7 | 29.2 | 21.03 | 36.6 | Below | 38.8 | Below | 38.6 | Below | NA | UD | NA | UD |
| | | | | | | | LLoD | | LLoD | | LLoD | | | | |

TABLE 5
viral RNA copy numbers in human plasma and PBM C specimens.

| | | PERV-A | | PERV-B | | PCMV | | PCV3 | | PLHV-1 | | PLHV-2 | | PLHV-3 | |
|---|---|---|---|---|---|---|---|---|---|---|---|---|---|---|---|
| | | | Copy | | Copy | | Copy | | Copy | | Copy | | Copy | | Copy |
| Cardiac | | | number | | number | | number | | number | | number | | number | | number |
| Xeno- | | | (Copies/ | | (Copies/ | | (Copies/ | | (Copies/ | | (Copies/ | | (Copies/ | | (Copies/ |
| trans- | | | ul of | | ul of | | ul of | | ul of | | ul of | | ul of | | ul of |
| plant | | | Plasma | | Plasma | | Plasma | | Plasma | | Plasma | | Plasma | | Plasma |
| Recip- | | | or | | or | | or | | or | | or | | or | | or |
| ient | | | Copies/ | | Copies/ | | Copies/ | | Copies/ | | Copies/ | | Copies/ | | Copies/ |
| Sam- | Time | | ng | | ng | | ng | | ng | | ng | | ng | | ng |
| ples | points | Ct | RNA) | Ct | RNA) | Ct | RNA) | Ct | RNA) | Ct | RNA) | Ct | RNA) | Ct | RNA) |

Plasma

| | | | | | | | | | | | | | | | |
|---|---|---|---|---|---|---|---|---|---|---|---|---|---|---|---|
| Xeno | Pre- | NA | UD | NA | UD | NA | UD | NA | UD | NA | UD | 35.43 | Below | NA | UD |
| Rec | Trans- | | | | | | | | | | | | LLoD | | |
| #1 | plant | | | | | | | | | | | | | | |
| | Day 6 | NA | UD | NA | UD | NA | UD | NA | UD | NA | UD | 35.59 | Below | NA | UD |
| | | | | | | | | | | | | | LLoD | | |
| | Day 28 | NA | UD | NA | UD | NA | UD | NA | UD | NA | UD | 35.56 | Below | NA | UD |
| | | | | | | | | | | | | | LLoD | | |
| Xeno | Pre- | NA | UD | NA | UD | NA | UD | NA | UD | NA | UD | 35.33 | Below | NA | UD |
| Rec | Trans- | | | | | | | | | | | | LLoD | | |
| #2 | plant | | | | | | | | | | | | | | |
| | Day 7 | NA | UD | NA | UD | NA | UD | NA | UD | NA | UD | 35.12 | Below | NA | UD |
| | | | | | | | | | | | | | LLoD | | |
| | Day 30 | NA | UD | NA | UD | NA | UD | NA | UD | NA | UD | 35.35 | Below | NA | UD |
| | | | | | | | | | | | | | LLoD | | |

PBMC

| | | | | | | | | | | | | | | | |
|---|---|---|---|---|---|---|---|---|---|---|---|---|---|---|---|
| Xeno | Pre- | NA | UD | NA | UD | NA | UD | NA | UD | NA | UD | 35.17 | Below | NA | UD |
| Rec | Trans- | | | | | | | | | | | | LLoD | | |
| #1 | plant | | | | | | | | | | | | | | |
| | Day 6 | NA | UD | NA | UD | 37.51 | Below | NA | UD | NA | UD | 35.13 | Below | NA | UD |
| | | | | | | | LLoD | | | | | | LLoD | | |
| | Day 28 | NA | UD | NA | UD | NA | UD | NA | UD | NA | UD | 35.15 | Below | NA | UD |
| | | | | | | | | | | | | | LLoD | | |
| Xeno | Pre- | NA | UD | NA | UD | NA | UD | NA | UD | NA | UD | 35.29 | Below | NA | UD |
| Rec | Trans- | | | | | | | | | | | | LLoD | | |
| #2 | plant | | | | | | | | | | | | | | |
| | Day 7 | NA | UD | NA | UD | NA | UD | NA | UD | NA | UD | 35.06 | Below | NA | UD |
| | | | | | | | | | | | | | LLoD | | |
| | Day 30 | NA | UD | NA | UD | NA | UD | NA | UD | NA | UD | 34.95 | Below | NA | UD |
| | | | | | | | | | | | | | LLoD | | |

### 4. Discussion

Xenotransplant studies involving organs such as heart, kidney and pancreatic islet cells from GEP-to-NHP models emphasized the utility of xenotransplant to overcome the shortage of organ donors (Kuwaki et al., Nat Med, (2015), 11:29-31; Yamada et al., Nat Med, (2005), 11:32-4; Yamamoto et al., Transplantation, (2019), 103:2090-104; Hering et al., Nat Med, (2006), 12:301-3; Mohiuddin et al., Am J Transplant, (2012), 12:763-71; Shin et al., Am J Transplant, (2015), 15:2837-50; Sun et al., J Clin Invest, (1996), 98:1417-22; van der Windt et al., Am J Transplant, (2009), 9:2716-26). Nevertheless, with the potential clinical benefit, a unique challenge in porcine xenotransplant became obvious, the possibility of transmission of porcine viruses to the recipients. Testing of the presence of porcine viruses in human specimens was not required until now. However, with the increasing popularity of xenotransplant with success stories of GEP-to-Human cardiac xenotransplant at the University of Maryland, Baltimore this testing procedure became essential (Griffith et al., N Engl J Med, (2022), 387:35-44). Among various porcine viruses some retroviruses and herpesviruses have higher risk of possible human transmission due to long latency. Hence, we had developed real-time qPCR based assays to detect and quantify PERV-A, PERV-B, PCMV, PCV3, PLHV-1, PLHV-2, and PLHV-3 viral RNAs in plasma and PBMC specimens at our center for regular surveillance of at-risk persons that include the xenotransplant recipients and the health care providers working with the recipients.

Our real-time qPCR-based assays were robust, sensitive, and specific for all the 7 porcine viruses tested, with a large range of detection limits. All the primers could detect the respective in-house positive controls and no non-specific amplification were detected from human gDNA or positive controls of other porcine viruses. In silico alignment analysis also confirmed the specificity of the primer/probe sets for porcine viruses only. We found no significant sequence similarities of these primers/probes with any human retroviruses. In the real-time qPCR assays, we have used SY BR-green chemistry for the detection of PERV-A and PERV-B and TaqM an chemistry for PCMV, PCV3, PLHV-1, PLHV-2 and PLHV-3. Since we did not have access to seropositive donor pigs to test new primer/probe sets for all the viruses, we used primers/probes that were tested previously in seropositive pigs by other study groups (Mueller et al., J Virol, (2002), 76:4734-40; Wang et al., J Virol Methods, (2017), 248:177-80; McMahon et al., Vet Microbiol, (2006), 116:60-8; Bosch et al., J Virol, (2000), 74:8575-81; Mazurek et al., Curr Microbiol, (2013), 67:505-14; Lu et al., Front Microbiol, (2022), 13:840347). Standards of known copy number is the most essential component for accurate quantification of the viral nucleic acids. While commercially available PK-15 cells can be used as standards/positive controls for PERV-A, PERV-B, there is lack of readily available standards/positive controls for PCMV, PCV3, PLHV-1, PLHV-2 and PLHV-3. Therefore, we have generated in house plasmid positive controls for these viruses which can be used as standards to quantify the respective porcine viruses. Using these plasmids we have rigorously tested the LL OD for the respective viruses and shown a large range of detection limits (ranging from 10 copies/μL to 10⁸ copies/μL).

We have used these assays to verify the presence of these 7 porcine viruses in donor porcine tissue samples and tested possible transmission in human PBM C and serum samples from xenotransplant recipients. PERV-A and PERV-B is known to be present in the genome of all pig strains with varying copy numbers (Patience et al., J Virol, (2001), 75:2771-5; Denner, J., Xenotransplantation, (2011), 18:151-7; Lopata et al., Front Microbiol, (2018), 9:730). Presence of PERV-A and PERV-B in the donor pig PBMCs was known before the xenotransplant (Griffith et al., N Engl J Med, (2022), 387:35-44). Our assays detected the viral RNAs in low quantity for PERV-A and PERV-B in heart, liver, spleen and lung of the donor pig tissues indicating the effective use of the assay. No other porcine viral RNAs were detected in any of the pig donor organs. When tested in human specimens, our assays confirmed no transmission of porcine viruses in human blood specimens following one month of xenotransplants. It would be appropriate to test the applicability of these assays in human test subjects who are positive for the porcine viruses, but we could not do that for obvious reasons, as no human subject is known to be naturally infected with these porcine viruses till date.

In conclusion, we believe that the assays we developed and tested in our lab will serve as a reliable and robust method for regular surveillance of at-risk persons involved in GEP-to-human xenotransplant.

What is claimed is:

1. A method of detecting and quantifying the presence of porcine viruses in a sample from a xenotransplant subject, comprising,
i) providing a sample from the xenotransplant subject, wherein the subject has received cells, tissues or an organ from a pig;
ii) detecting the presence or absence of porcine virus nucleic acid in the sample that is amplified using a polymerase chain reaction (PCR) assay; and
iii) quantifying porcine virus nucleic acid detected in the sample, wherein the porcine virus comprises one or more of PERV-A, PERV-B, PCMV, PCV3, PLHV-1, PLHV-2, and PLHV-3.

2. The method of claim 1, wherein the cells, tissue or organ has been genetically modified.

3. The method of claim 1, wherein the cells, tissue or organ has been genetically modified to attenuate rejection by the subject.

4. The method of claim 1, wherein the method comprises detecting the presence of absence of nucleic acid from PERV-A, PERV-B, PCMV, PCV3, PLHV-1, PLHV-2, and PLHV-3.

5. The method of claim 1, wherein the amplified nucleic acid is quantified using a dye that binds to the porcine virus nucleic acid.

6. The method of claim 5, wherein the dye is a fluorescent dye.

7. The method of claim 1, wherein the amplified nucleic acid is quantified using a labeled nucleic acid probe that binds to the nucleic acid.

8. The method of claim 7, wherein the probe is labeled with a fluorescent dye.

9. The method of claim 1, wherein copy number of the porcine virus nucleic acid in the sample is quantified using a standard curve generated using amplified control nucleic acid.

10. The method of claim 9, wherein the amplified control nucleic acid is generated by polymerase chain reaction with a plasmid template comprising the porcine virus nucleic acid of a known quantity.

11. The method of claim 1, wherein the subject is a human.

12. The method of claim 11, wherein the sample is selected from the group consisting of blood, plasma, and peripheral blood mononuclear cells (PBMCs).

13. The method of claim 1, wherein the amplified PERV-A and PERV-B nucleic acid is quantified using a fluorescent dye that binds to the nucleic acid.

14. The method of claim 1, wherein the amplified PCMV, PCV3, PLHV-1, PLHV-2, and PLHV-3 nucleic acid is quantified using a nucleic acid probe labeled with a fluorescent dye.

15. The method of claim 9, wherein the lower limit of detection of the copy number of PERV-A, PERV-B and PLHV-2 is about 25 copies/μL.

16. The method of claim 9, wherein the lower limit of detection of the copy number of PCMV, PCV3, PLHV-1 and PLHV-3 is about 10 copies/μL.

17. The method of claim 1, wherein the number of cycles of PCR is thirty-six (36) or less.

18. The method of claim 1, wherein PCMV nucleic acid is amplified using a forward oligonucleotide primer comprising SEQ ID NO:1 and a reverse oligonucleotide primer comprising SEQ ID NO:2.

19. The method of claim 1, wherein PCV3 nucleic acid is amplified using a forward oligonucleotide primer comprising SEQ ID NO:3 and a reverse oligonucleotide primer comprising SEQ ID NO:4.

20. The method of claim 1, wherein PLHV-1 nucleic acid is amplified using a forward oligonucleotide primer comprising SEQ ID NO:5 and a reverse oligonucleotide primer comprising SEQ ID NO:6.

21. The method of claim 1, wherein PLHV-2 nucleic acid is amplified using a forward oligonucleotide primer comprising SEQ ID NO:7 and a reverse oligonucleotide primer comprising SEQ ID NO:8.

22. The method of claim 1, wherein PLHV-3 nucleic acid is amplified using a forward oligonucleotide primer comprising SEQ ID NO:9 and a reverse oligonucleotide primer comprising SEQ ID NO:10.

23. The method of claim 1, wherein PERV-A nucleic acid is amplified using a forward oligonucleotide primer comprising SEQ ID NO:11 and a reverse oligonucleotide primer comprising SEQ ID NO:12.

24. The method of claim 1, wherein PERV-B nucleic acid is amplified using a forward oligonucleotide primer comprising SEQ ID NO:13 and a reverse oligonucleotide primer comprising SEQ ID NO:14.

25. The method of claim 1, wherein PCMV nucleic acid is quantified using an oligonucleotide probe comprising SEQ ID NO:15.

26. The method of claim 1, wherein PCV3 nucleic acid is quantified using an oligonucleotide probe comprising SEQ ID NO:16.

27. The method of claim 1, wherein PLHV-1 nucleic acid is quantified using an oligonucleotide probe comprising SEQ ID NO:17.

28. The method of claim 1, wherein PLHV-2 nucleic acid is quantified using an oligonucleotide probe comprising SEQ ID NO:18.

29. The method of claim 1, wherein PLHV-3 nucleic acid is quantified using an oligonucleotide probe comprising SEQ ID NO:19.

30. The method of claim 1, wherein the subject has received a genetically modified pig heart.
